(19) United States
(12) Patent Application Publication
(10) Pub. No.: US 2026/0160738 A1
(43) Pub. Date: Jun. 11, 2026

(54) SYSTEM, METHOD, AND APPARATUS TO SUPPORT ON-SITE CONCRETE INSPECTION

(71) Applicant: Gecko Robotics, Inc, Pittsburgh, PA (US)

(72) Inventors: Weronika van Vianen, Philadelphia, PA (US); Olga Suchankova, New York, NY (US); Himal King, Brooklyn, NY (US); Christopher Connor Hazen, Placerville, CO (US); Domenic P. Rodriguez, Pittsburgh, PA (US); Lincoln Roop, Pittsburgh, PA (US); Michael A. Binger, Pittsburgh, PA (US); Edward A. Bryner, Pittsburgh, PA (US)

(21) Appl. No.: 19/409,505

(22) Filed: Dec. 4, 2025

Related U.S. Application Data
(63) Continuation of application No. PCT/US2024/033270, filed on Jun. 10, 2024.
(60) Provisional application No. 63/471,874, filed on Jun. 8, 2023.
(60) Provisional application No. 63/507,945, filed on Jun. 13, 2023.

(51) Int. Cl. G01N 29/265; G01B 17/02; G01N 29/07; G01N 33/38; G05D 1/689; G05D 105/80

(52) U.S. Cl. CPC G01N 29/265; G01B 17/02; G01N 29/07; G01N 33/383; G05D 1/689; G01N 2291/011; G01N 2291/0232; G01N 2291/02854; G01N 2291/0289; G05D 2105/89

(57) ABSTRACT

A device may include an inspection robot comprising a payload configured to interrogate an inspection surface in response to the inspection robot moving on the inspection surface, the payload comprising an ultrasonic (UT) sensor. A device may include a data verification controller, comprising: a verification data circuit structured to interpret verification data from the payload, an inspection status circuit structured to determine an inspection data quality value in response to the verification data; and a data verification circuit structured to perform a data verification operation in response to the inspection data quality value.

### CROSS-REFERENCE TO RELATED APPLICATIONS

This application claims benefit of and is a continuation of International Patent Application No. PCT/US 2024/033270 (Attorney Docket No. GROB-0023-WO), filed Jun. 10, 2024, and entitled “SYSTEM, METHOD, AND APPARATUS TO SUPPORT ON-SITE CONCRETE INSPECTION,” International Pub. No. WO 2024/254597, which is hereby incorporated by reference in its entirety for all purposes.

International Patent Application No. PCT/US2024/033270 claims the benefit of U.S. Provisional Patent App. No. 63/507,945, filed on Jun. 13, 2023, entitled “INSPECTION ROBOT FOR CONCRETE QUALITY INSPECTION” (Attorney Docket No. GROB-0026-P01); and claims the benefit of U.S. Provisional Patent App. No. 63/471,874, filed on Jun. 8, 2023, entitled “INSPECTION ROBOT WITH MODULAR COMPONENTS AND HIGH CONFIGURABILITY” (Attorney Docket No. GROB-0022-P01).

Each of the foregoing applications is incorporated herein in the entirety for all purposes.

### BACKGROUND

Previously known systems suffer from a number of challenges, for example ensuring that the correct sensor package is prepared for the inspection surface, ensuring that the data collection parameters are properly configured (e.g., impactor commands, transducer reading operations, collection of data, processing parameters for the inspection data, etc.), and ensuring that the sensors as installed are properly able to get effective readings from the surface.

### SUMMARY

Embodiments of the present disclosure provide for a number of improvements to concrete inspection operations, for example using automated inspection operations of a surface that is bonded to (or ideally bonded to) concrete. Example embodiments include a ferrous surface (e.g., a steel surface) with a concrete wall or floor backing the surface.

Inspection operations involve the inspection robot proceeding over the surface unsupervised for extended periods, and the costs of determining that significant portions of the data are not correctly collected after the fact are significant. Further, the inspection operations and related processing are complex, putting a high operational burden on the operator on-site to perform data analysis and ensure the inspection will be successful before commencing.

Embodiments herein provide for systems and procedures to perform automated data validation, to ensure that the inspection robot is properly configured, and to rapidly identify any deviations to allow the operator to intervene and mitigate down time or other related costs. Embodiments herein provide for systems and procedures to allow the operator to adjust processing parameters in real time, including time windows related to the processing, and/or any other parameters such as data quality cutoffs, material properties of the inspection surface, or the like. Embodiments herein provide for convenient and high capability analysis of the inspection data, both during run-time operations and/or as a post-processing operation, allowing the operator or other user to rapidly confirm that the inspection is complete and sufficient, to check and configure processing parameters and determine their effects on data quality, and/or to rapidly identify and characterize anomalies found during inspection operations.

### BRIEF DESCRIPTION OF THE FIGURES

FIG. 1 is a schematic diagram of a system to support on-site concrete inspection.

FIG. 2 is a schematic diagram of a controller to support on-site concrete inspection.

FIG. 3 schematically depicts a number of illustrative data quality value considerations.

FIG. 4 is a schematic depiction of a user interface according to embodiments herein.

FIG. 5 is an illustrative inspection data display.

FIG. 6 is an illustrative inspection data display.

FIG. 7 is a schematic depiction of a user interface according to embodiments herein.

FIG. 8 schematically depicts a number of illustrative inspection data map options.

FIG. 9 is a schematic depiction of a procedure to perform data verification.

FIG. 10 is an illustrative inspection data display.

FIG. 11 is a schematic diagram of a controller to support on-site concrete inspection.

FIG. 12 is a schematic depiction of a procedure to adjust a window processing parameter.

FIG. 13 is a schematic diagram of a controller to support on-site concrete inspection.

FIG. 14 schematically depicts a number of illustrative pre-check workflow operations.

FIG. 15 is a schematic depiction of a procedure to enforce a pre-check workflow operation.

FIG. 16 is a schematic diagram of a controller to support on-site concrete inspection.

FIG. 17 is a schematic depiction of a procedure to adjust a UT processing parameter.

FIG. 18 is a schematic diagram of a controller to support concrete inspection operations.

FIG. 19 is a schematic diagram of a procedure to support concrete inspection operations.

FIG. 20 is an illustrative inspection data display.

FIG. 21 schematically depicts a number of inspection displays.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles disclosed herein as would normally occur to one skilled in the art to which this disclosure pertains.

Embodiments herein reference an inspection robot as a baseline term to describe a robot that can support any of these operations, including a subset of these operations, or all of these operations, for clarity of the present description. The specific operations performed may nevertheless not be “inspection” operations in certain configurations and/or while performing certain operations. Similarly, embodiments herein reference an inspection surface as a baseline term to describe a service location, and specifically the portion of the service location that is engaged by the inspection robot. An inspection surface, in certain embodiments, may be a serviced portion of the location, whether the specific service(s) performed include(s) inspection, visualization, marking, cleaning, and/or repair. Example and non-limiting inspection surfaces include, without limitation, surfaces such as: a tank wall; a pipe wall; a surface associated with any industrial process or equipment; a cooling tower; a pressure vessel; a tray or interior feature; and/or a heat transfer tube, wall, pipe, or the like. In certain embodiments, an inspection surface may include a metallic surface and/or a ferrous surface. Example inspected surfaces may include any exterior or interior surface, an elevated surface (e.g., a surface including at least a portion that is at a relevant height for fall protection considerations), and/or a confined space (e.g., a surface including at least a portion that would be considered a confined space).

In certain embodiments, an operation may be understood to be an inspection operation for one purpose, but another type of operation for another purpose (e.g., a visualization operation of the surface may be understood to be an inspection operation, but may additionally or alternatively be a preparatory operation, a confirmation operation, etc., which may depend upon the entity describing the operation, whether any anomalies and/or features are detected during the operation, etc.). The specific terminology utilized for an operation is not limiting to the present disclosure, and “inspection operations” or similar terminology utilized herein should be understood to include any service operations, performable by inspection robots set forth herein, at a service location.

Embodiments of the present disclosure may be utilized with various inspection robots that perform inspection operations on highly complex data, including automated inspection operations that are performed with limited human intervention during inspection operations. Example and non-limiting inspection robots that may be utilized herewith include, without limitation, inspection robots as set forth in one or more of the following patents or patent applications: U.S. Pat. No. 10,698,412, filed on 22 Dec. 2017, Entitled “inspection Robot With Couplant Chamber DISPOSED WITHIN SLED FOR ACOUSTIC COUPLING” (GROB-0003-U01); U.S. Pat. No. 11,673,272, filed on 9 Mar. 2020, entitled “INSPECTION ROBOT WITH STABILITY ASSIST DEVICE” (GROB-0007-U01); and/or U.S. Pat. No. 11,865,698, filed on 8 Apr. 2022, entitled “INSPECTION ROBOT WITH REMOVEABLE INTERFACE PLATES AND METHOD FOR CONFIGURING PAYLOAD INTERFACES” (GROB-0010-U01). Each one of the foregoing patents and/or patent applications is incorporated herein by reference in the entirety for all purposes.

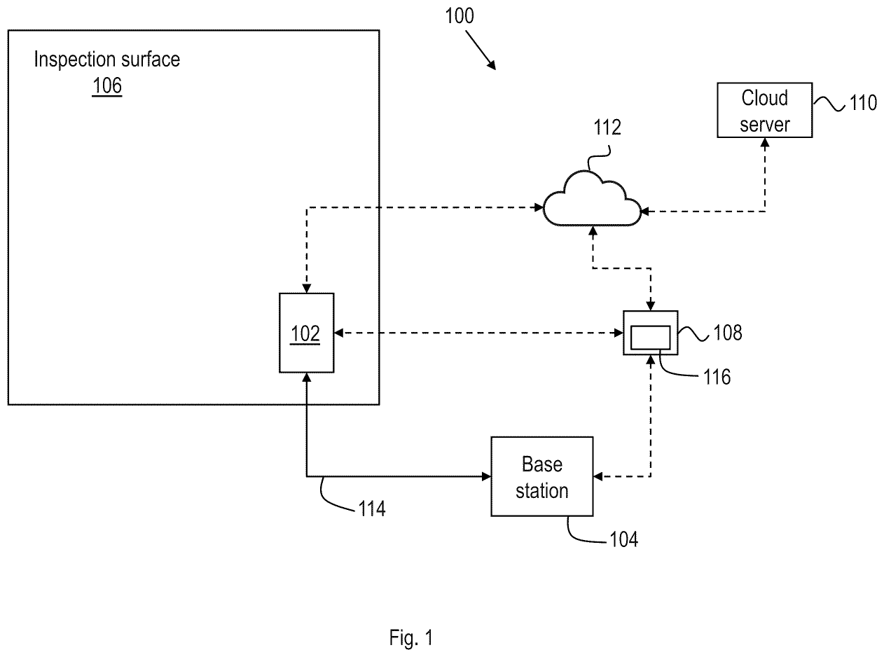

Referencing FIG. 1, an example system 100 is schematically depicted, having an inspection surface 106 (e.g., a surface of a tank, pipe, industrial equipment, etc., that may include a concrete surface, for example backing a metallic surface). The example system 100 includes an inspection robot 102 having a payload with an ultrasonic (UT) sensor mounted thereon, where the inspection robot traversing the surface performs inspection operations by interrogating the inspection surface 106 with the UT sensor(s), for example by passing over the surface with multiple UT sensors, and/or by rastering one or more UT sensors across the inspection surface 106. The example system 100 includes a base station 104, for example providing power and couplant to the inspection robot 102 (e.g., using a tether 114). In certain embodiments, the base station 104 additionally or alternatively provides communicative support for the inspection robot 102, passing communications on to an operator user device 108 and/or to a cloud server 110. In certain embodiments, the base station 104 includes the user device 108 (e.g., as a computing device mounted with the other aspects of the base station). In certain embodiments, the inspection robot 102 communicates directly to the user device 108 and/or the cloud server 110. In certain embodiments, the cloud server 110, where present, communicates to the user device 108, the inspection robot 102, and/or the base station 104 utilizing the internet 112, a WAN, or utilizing any other communication scheme.

The example system 100 includes a controller 202 having a number of components thereof that are configured to functionally execute operations of the controller 202. The example controller 202 is depicted as a single device for clarity of the present description, but the controller 202 may be a distributed device, in whole or part, and/or the distribution of the controller 202 may be varied at certain operating conditions and/or during certain operations. For example, aspects of the controller 202 may be embodied on the user device 108, in the cloud server 110, on the inspection robot 102, and/or on the base station 104. In certain embodiments, components of the controller 202, for example including circuits, modules, interfaces, or the like, may be embodied as any sensor, actuator, computing device, computer readable instructions stored on a non-transient media and configured to perform one or more operations of the computer in response to a processor executing the instructions, logic circuits, display devices, input/output devices, or the like.

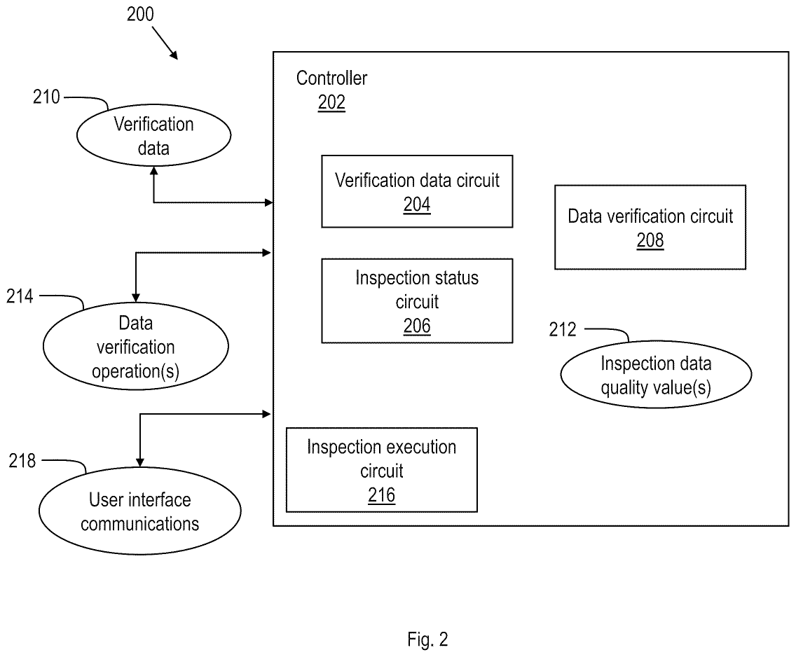

Referencing FIG. 2, an example apparatus 200 includes a controller 202, embodied as a data verification controller, having a verification data circuit 204 that interprets verification data 210 from the payload, an inspection status circuit 206 that determines an inspection data quality value 212 in response to the verification data 210, and a data verification circuit 208 that performs a data verification operation (e.g., executed utilizing data verification operation(s) 214, for example providing commands, requesting data, and/or providing communications with any aspect of the system 100, such as the inspection robot 102, user device 108, and/or cloud server 110), in response to the inspection data quality value 212. The example verification data 210 includes any inspection data and/or metadata related to the inspection data that is utilized according to any operations set forth herein to verify that collected data is properly collected and representative of the inspection surface.

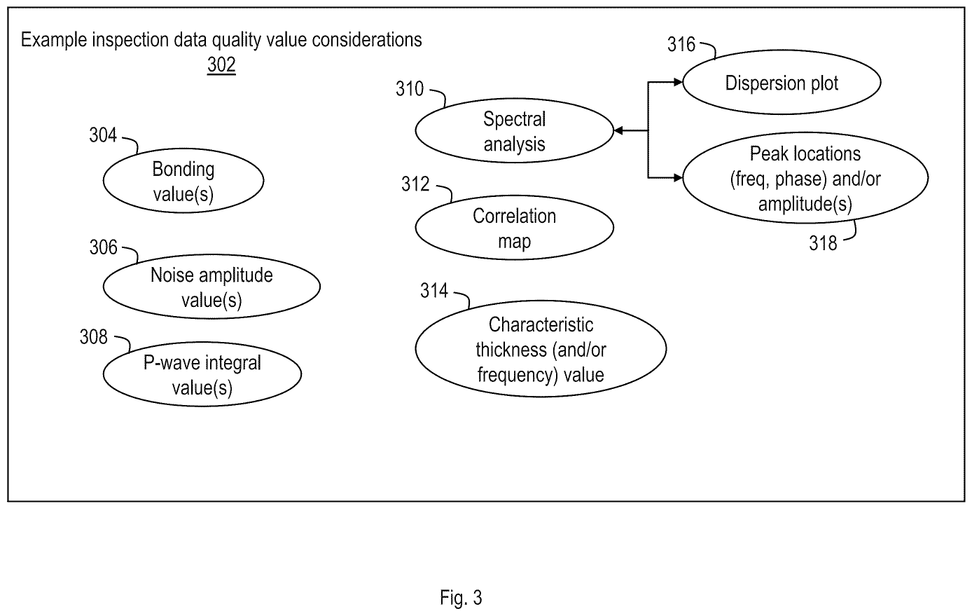

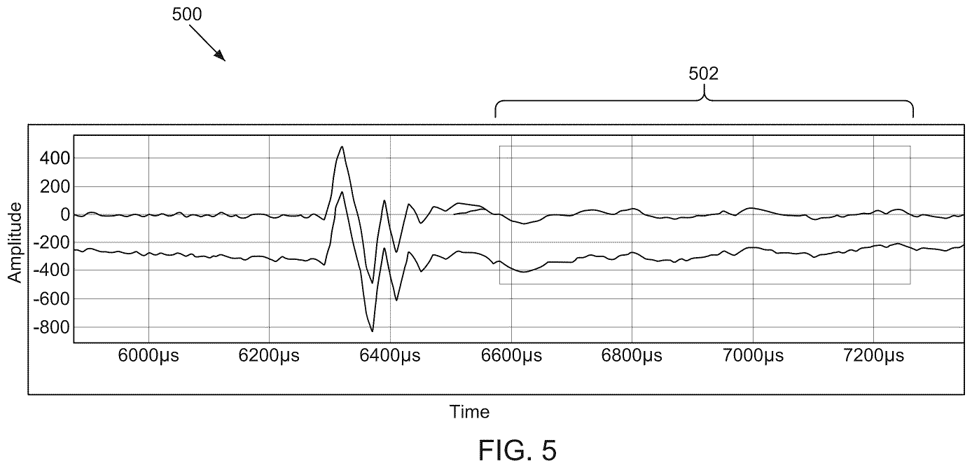

Referencing FIG. 3, example and non-limiting considerations 302 are schematically depicted for determining the inspection data quality value 212. An example inspection status circuit 206 determines the inspection data quality value 212 by determining a bonding value 304—for example determining that the data quality is acceptable where the bonding value 304 indicates a good bond with the concrete, and determining that the data quality is not acceptable, and/or marking the data, where the bonding value 304 indicates a poor bond with the concrete. In certain embodiments, the bonding value 304 can be determined according to the amplitudes in the scans (e.g., well bonded surface should dampen the response), according to anomalies and/or specific patterns in a dispersion plot, or the like. An example inspection status circuit 206 determines the inspection data quality value 212 by determining a noise amplitude value 306, for example by observing the noise level during a period where the impactor is not active, to determine the baseline noise in the reading at that location. In certain embodiments, the noise amplitude value 306 may additionally or alternatively include determining a noise ratio - for example based on the integrated amplitude of the noise and the signal (e.g., integrated absolute value of the P-wave, reference FIG. 5 and the related description), to allow determination of the inspection data quality value 212 based on the relevant signal to noise ratio. An example inspection status circuit 206 determines the inspection data quality value by determining a P-wave integral value 308 (e.g., determining the amount of energy sent and returned, comparing signal return energy to noise in the system, and/or determining whether the P-wave integral 308 indicates the data value is an outlier or an anomaly may be present).

An example inspection status circuit 206 determines the inspection data quality value in response to a correlation map 312. An example correlation map may utilize an expected Gaussian distribution and Gaussian correlation metric, although any correlation metric may be utilized (e.g., a gradient determination, moving average check between data values, etc.). The correlation map 312 provides a visual depiction of where data points tend to be more distinct than surrounding data points, which can be useful to determine feature locations, damage locations, or the like, and the trajectory of the changes on the correlation map can additionally provide signatures that can be utilized to identify the type of feature, anomaly, or damage present at the location.

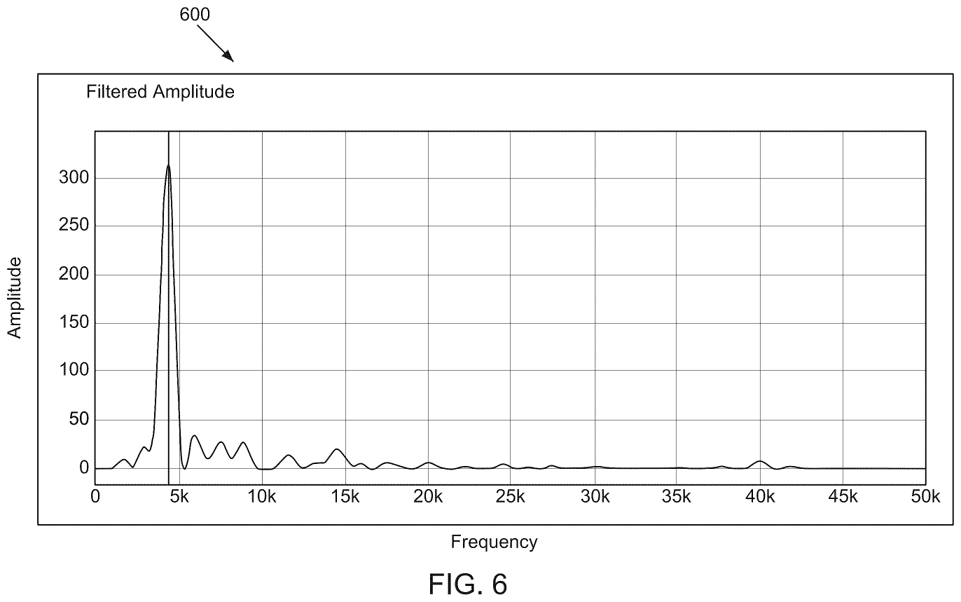

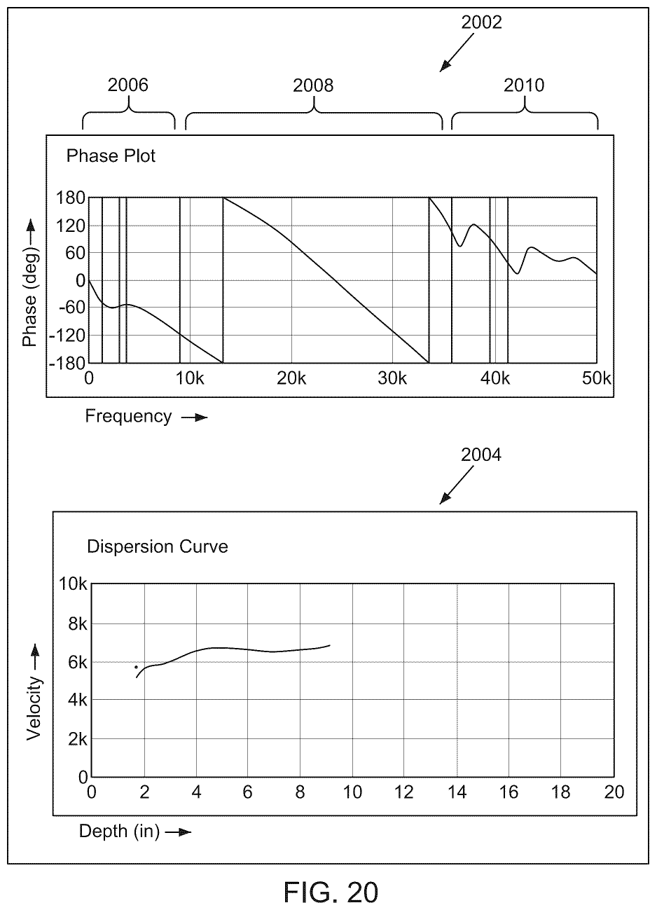

An example inspection status circuit 206 determines the inspection data quality value in response to a characteristic thickness value 314. For example, the frequency of a dominant signal (e.g., reference FIG. 6) can be converted into a characteristic thickness, allowing for the determination of indicated thickness of the asset, confirmation of the layers (e.g., ferrous surface, concrete substrate, etc.), and/or parsing the data to determine anomaly types (e.g., voids, material loss, honeycombing, etc.). In certain embodiments, the characteristic thickness value 314 combined with a depth exploration such as the dispersion curve (e.g., reference FIG. 20 and the related description), to determine the overall data quality value, as well as support feature identification and various inspection displays as set forth herein.

An example inspection status circuit 206 determines the inspection data quality value in response to a spectral analysis 310 of the verification data. In certain embodiments, the spectral analysis 310 allows for checking the position and amplitude of various frequency peaks 318 in the signal, and the location (frequency) and/or strength (e.g., amplitude) of the frequency peaks may be utilized to determine whether the data is properly collected and/or to identify features and/or anomalies. In certain embodiments, the frequency analysis can be utilized to create a phase plot (e.g., reference FIG. 20) and a dispersion plot 316, as a part of determining the inspection data quality, as well as support feature identification and various inspection displays as set forth herein.

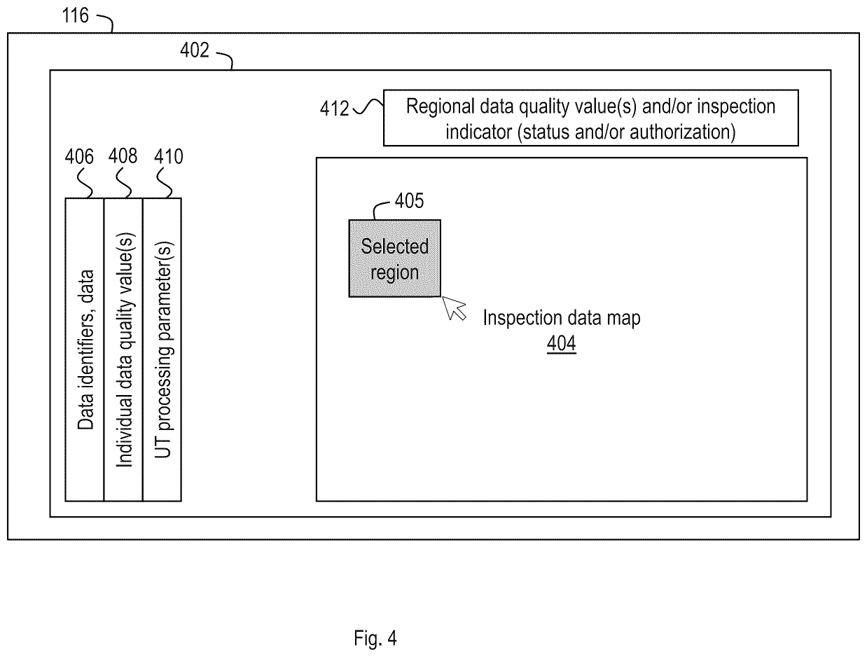

In certain embodiments, a data verification operation 214 includes labeling a range of data values with the inspection data quality value. For example, referencing FIG. 4, an example user interface 116 (e.g., implemented by an inspection execution circuit 216, interacting via user interface communications 218) includes a data validation interface 402 showing an inspection data map 404 (e.g., a display of inspection data overlaid on a portion of a map of the asset having he inspection surface 106), and in the example a portion of the data is graphically selected as a selected region 405. In the example of FIG. 4, the underlying data for the selected region 405 is depicted in a table at the left side of the interface 402, with a column 406 for identifying data (e.g., a time stamp, asset location, metadata, etc. for the particular data point), an individual data quality value 408 (e.g., an indicator of whether the particular data point is likely to be valid collected data), and a regional data quality value 412. In certain embodiments, the quality values may be color coded (e.g., green is good, red is not good, yellow is suspect or lower confidence data), a numerical value (e.g., utilizing a formula between various inputs to create an indexed value), or the like. In certain embodiments, threshold values may be utilized for individual data point quality determination (e.g., the selected time window does/does not clip signal data, the amplitudes indicate good concrete bonding, the operations of the impactor appeared to be proper from a peak voltage and deadband perspective, etc.), and aggregated values, averages, or the like, may be utilized for regional data point quality determination and/or for overall inspection coverage and/or data quality determinations. For example, a cutoff of no more than 2% of data points being clipped, or no more than 1% of data points outside of the deadband, may be utilized to determine the overall data quality for a regional data point quality determination. In certain embodiments, various thresholds may be set for a parameter, with determinations from the various thresholds being combined into an overall data point quality score or value.

In certain embodiments, the interface 402 may depict only one of the individual data quality values 408 or the regional data quality value 412. In certain embodiments, the regional data quality value 412 may depict the data quality of the entire depicted portion of the asset, for the entire asset, for the selected region 405, for a region associated with a feature of interest that is within the inspection data map 404 and/or proximate to the selected region 405, and/or for an inspection operation (e.g., providing an indicator of whether the inspection operation should be continued or paused to correct anything). In certain embodiments, operations by the user to group data values may be utilized to determine the scope of the regional data quality value 412, for example where the user can select data from the table, from the inspection data map 404, by filtering for certain data values and/or metadata values, or the like, and rapidly determine the inspection data quality for the selected group of data values. In certain embodiments, the overall data quality value may be determined according to the fraction of data values that can be utilized, according to coverage of the asset (e.g., inspection coverage relative to the planned inspection coverage), availability of good data points near enough to, and of sufficient density, near any features of interest, or the like. In certain embodiments, selected groups for data values can be labeled, for example according to the associated regional data quality value 412, and/or labeled by the user, with metadata for the label stored in association with the selected group, which may be utilized for reporting, auditing, further analysis, continuous improvement operations, or the like.

An example data verification operation includes providing an inspection status indicator, for example provided at 412 in the example of FIG. 4. The example inspection status indicator provides a single prominent location for the operator to determine whether the inspection data quality is sufficient, and/or to ensure that any checklists or workflows are complete before beginning or continuing inspection operations. In certain embodiments, the inspection status indicator allows for multiple persons to stay advised on the inspection status (e.g., by providing a separate communication to a user device, for example an off-site supervisor or analyst), and to receive a rapid notification if the inspection data quality has an issue. In certain embodiments, the inspection status indicator may additionally or alternatively be provided as a physical indicator on the inspection robot, for example where the inspection execution circuit 216 provides the inspection status indicator to the inspection robot, and a physical indicator on the inspection robot is responsive to the inspection status indicator (e.g., a green light if the inspection is approved or ongoing, and a red light if the inspection data quality is not sufficient). In certain embodiments, a light on the inspection robot is positioned such that it is visible to the operator in most conditions (e.g., depending on the form factor of the inspection surface), for example on a top or rear side of the inspection robot. In certain embodiments, the physical indicator includes a display on the inspection robot (e.g., on a top surface), where the display includes a light, button, graphical object, or the like providing the inspection status indicator. The example of FIG. 4 further includes a UT processing parameter 410 field, for example allowing the user to access, verify, and/or adjust UT processing parameters 410 of any type as set forth throughout the present disclosure.

Referencing FIG. 5, an example wave plot 500 is depicted, with illustrative data, that may be utilized in various analysis and user interface operations as set forth herein. The example plot includes a waveform from a typical transducer during collection operations, where the P-wave is analyzed during a time window 502, that begins as soon as possible after the initial returns, before the amplitude decays significantly in later cycles. The amplitude (and/or area under the curve) can be utilized to determine de-bonding, for example where bonding is good the concrete will dampen the signal, and where the bonding is lacking the signal characteristic will be evident in the amplitude wave plot 500. The time window 502 defines the portion of the signal that is processed as the P-wave, and the absolute area under the P-wave curve (e.g., integrated over the time window 502) is proportional to the energy returned, or the signal energy.

Referencing FIG. 6, an example peak plot 600 from a spectral analysis is depicted as illustrative data. The example peak plot 600 indicates the frequency and amplitude of components of the returns, and can be utilized to determine the characteristic frequency and accordingly the apparent thickness of the asset. The apparent thickness can be utilized to confirm that the data is of high value, to identify features such as voids or honeycombing, and to detect the strength of inputs such as noise into the data.

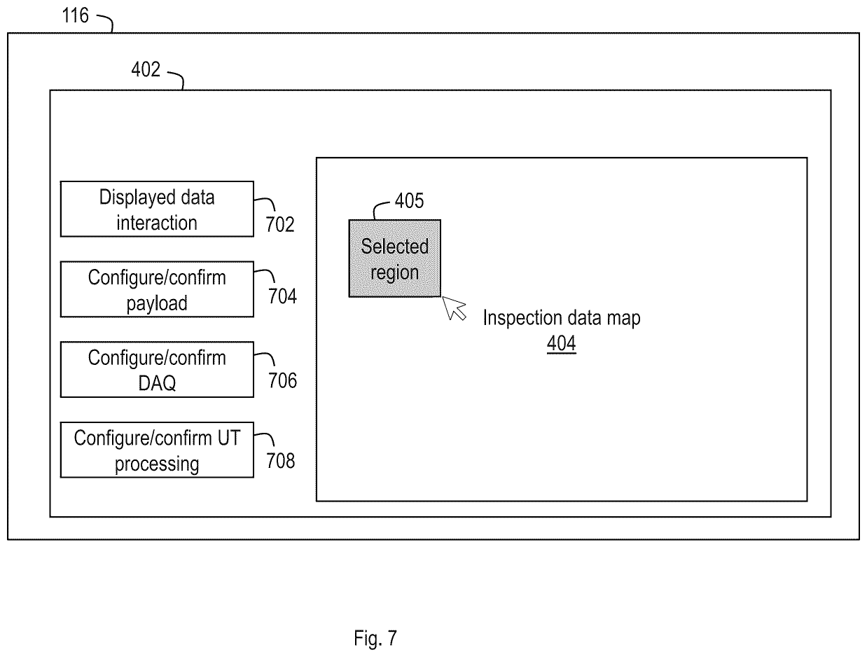

Referencing FIG. 7, an example user interface 116 is depicted, allowing a controller 202 to perform operations to support inspections, to enforce pre-check workflows, and/or to allow the user to identify and/or address low quality inspection data. The example user interface 116 includes an inspection data map 404, for example provided in a window adjustment interface 402 and/or a data analysis parameter interface 402, and provides components for displayed data interaction 702 (e.g., to select data for display, and/or to select which aspects are displayed), for confirming and/or configuring a payload 704 (e.g., to confirm which payload is mounted, to ensure the collection settings are correct for the asset, to ensure that the provided power and/or couplant is compatible, etc.), for confirming and/or configuring a data acquisition circuit (DAQ) 706 (e.g., ensuring the DAQ is compatible with the payload, and has the proper settings for collection, capture, and/or transmission of the data); and/or for confirming and/or configuring UT processing 708 (e.g., ensuring settings for excitement operations of the impactor(s), reading by the transducer(s), processing time windows, estimated material properties, etc., are set properly, and/or allowing for adjustment of these parameters).

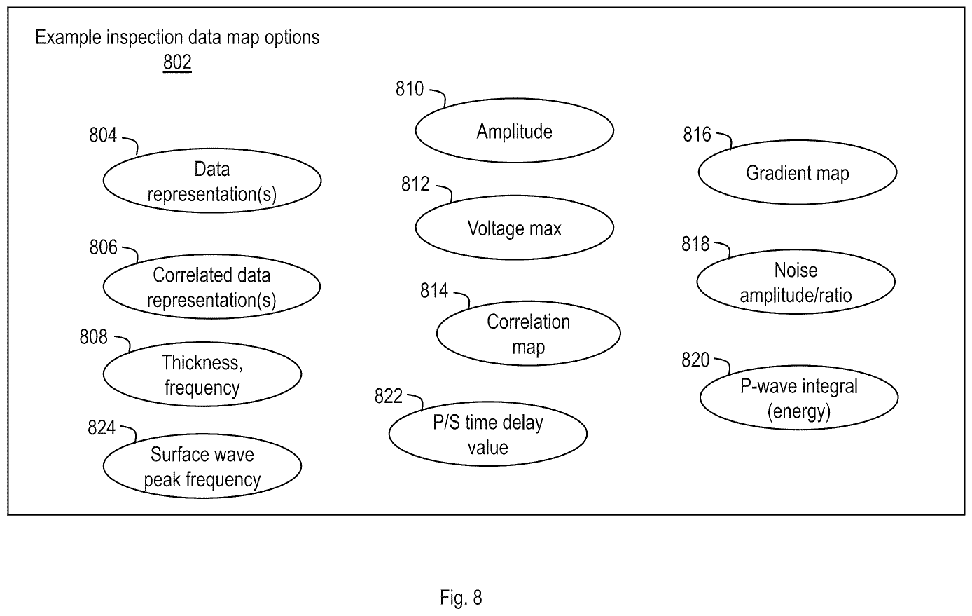

Referencing FIG. 8, and without limitation to any other aspect of the present disclosure, example and non-limiting inspection map options 802 are schematically depicted. The example options 802 include one or more of: data representations 804 (e.g., displaying inspection data on the asset, for example with numerical and/or color coding for data values and/or buckets of data values); correlated data representations 806 (e.g., representing data that is correlated from inspection data, for example asset thickness, wear value, time-to-service, etc.); a thickness and/or frequency value 808 (e.g., estimated thickness from observed frequencies, etc.); an amplitude value 810 (e.g., induced and/or received amplitude, and/or a corresponding voltage value); a voltage max 812 (e.g., maximum voltage during impactor operations); a correlation map 814 (e.g., depicting uniformity, gradients, anomalies, etc., on the inspection surface); a gradient map 816 (e.g., supporting the identification of certain features using a differential analysis); a noise amplitude/ratio 818 (e.g., depicting a noise amplitude and/or signal/noise ratio on the inspection surface); a P-wave integral value 820 (e.g., depicting signal energy received by location on the asset); a time delay value 822 (e.g., showing a time delay between arrival of a surface wave and the P-wave); and/or a surface wave peak frequency 824 (e.g., providing an indicator of poor bonding, a void, honeycombed substrate, etc.). In certain embodiments, the inspection data map options 802 include providing a dispersion plot on the user interface.

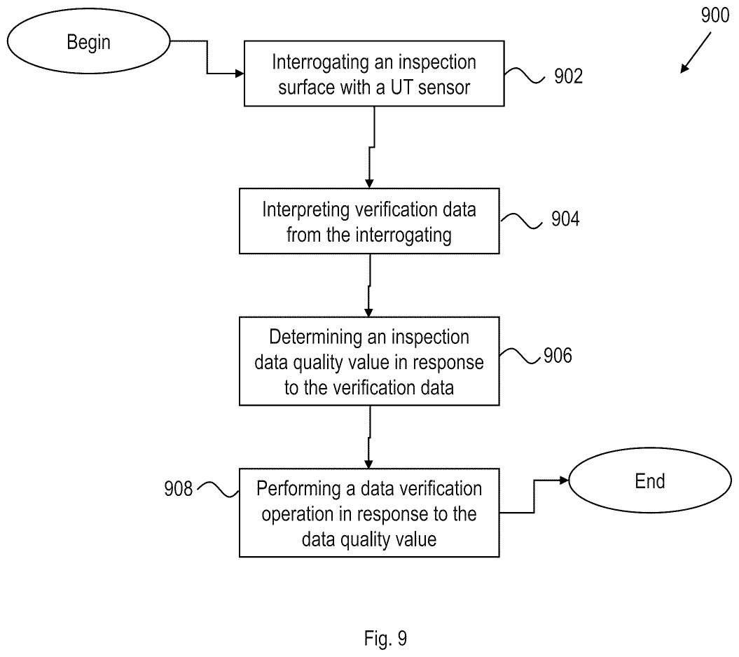

Referencing FIG. 9, an example procedure 900 includes an operation 902 to interrogate an inspection surface with a UT sensor, an operation 904 to interpret verification data from the interrogating, an operation 906 to determine an inspection data quality value in response to the verification data, and an operation 908 to perform a data verification operation in response to the data quality value. Procedures set forth throughout the present disclosure, including procedure 900, may be performed in whole or part by any controller, circuit, interface, system, or the like, as set forth throughout the present disclosure.

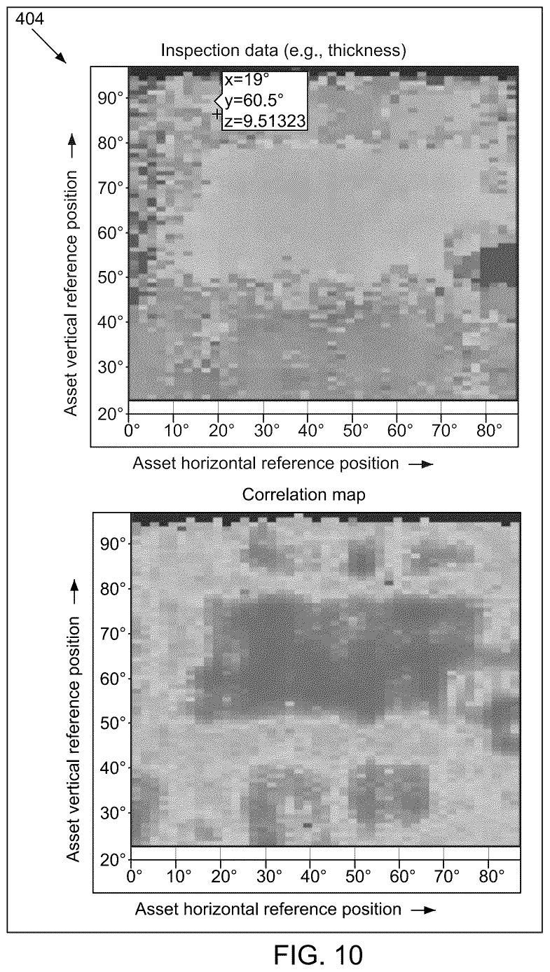

Referencing FIG. 10, an example inspection data map 404 depicts illustrative data, with inspection data (e.g., thickness, in the example) on the left portion of the display, and a correlation map (e.g., differential thickness using a Gaussian distribution, in the example) depicting the same region of the asset. The example of FIG. 10 allows for additional information to correlate data and determine the cause and description of an anomaly and/or data near a feature of interest. The correlation map provides a contrast to track transition regions, and further isolate the boundaries of features of the inspection surface.

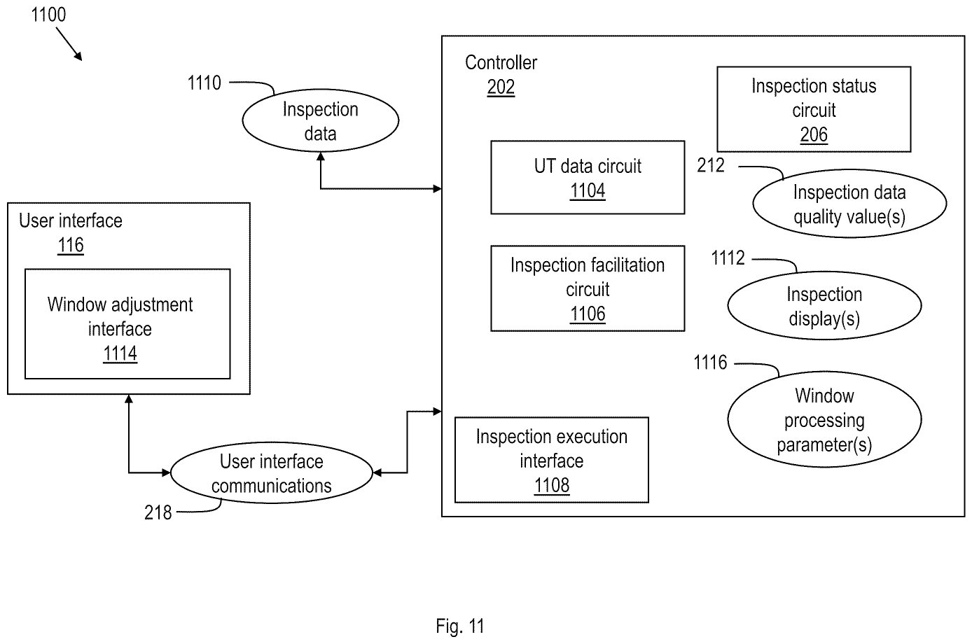

Referencing FIG. 11, an example apparatus 1100 includes a controller 202, that may be utilized with a system 100, and/or combined in whole or part: an ultrasonic (UT) data circuit 1104 structured to interpret UT inspection data 1110 from a payload of an inspection robot 102, the payload including an ultrasonic (UT) sensor; an inspection facilitation circuit 1106 structured to provide an inspection display 1112 on a user interface 116; an inspection execution interface 1108 structured to implement the user interface 116, to provide a window adjustment interface 1114 on the user interface 116, and to determine at least one window processing parameter 1116 in response to user interactions with the window adjustment interface 1114. The example apparatus 1100 allows the user to rapidly check, confirm, and/or adjust the window processing parameters utilized to determine the P-wave from a wave plot (and/or analytically from the data set), to confirm the inspection results and/or data quality in response to adjustments to the window processing parameters, and/or run scenarios to confirm that the inspection results are robust, and/or to generate illustrations of features or views of the inspection data that may be of interest to other users (e.g., an operator of a facility including the inspection surface).

Again referencing FIG. 5, an example window adjustment interface 1114 allows for the user to perform a drag and drop operation, e.g., on the box bounding the P-wave in the time window 502, to adjust the start time and/or width (e.g., measuring time window) of the P-wave analysis. In certain embodiments, the inspection display is updated to reflect the inspection results utilizing the window processing as adjusted in the window adjustment interface 1114, including potentially updating any quality determinations. An example inspection facilitation circuit 1106 updates the inspection display by providing a comparison display on the user interface - for example displaying the inspection display with a before and after view based on the window adjustments (e.g., side-by-side, such as in FIG. 5), and/or on a same display location (e.g., allowing the user to flip back and forth between the two, or more, scenarios). In certain embodiment, the inspection data quality value and/or inspection indicator(s) are updated on the user interface 116, for example by updating the inspection display, for example notifying the user that the inspection operations are ready to commence and/or proceed in response to window adjustments bringing the inspection data quality value to an acceptable level. An example inspection facilitation circuit 1106 allows the operator or user to adjust the window processing parameters for different values at different regions of the inspection surface.

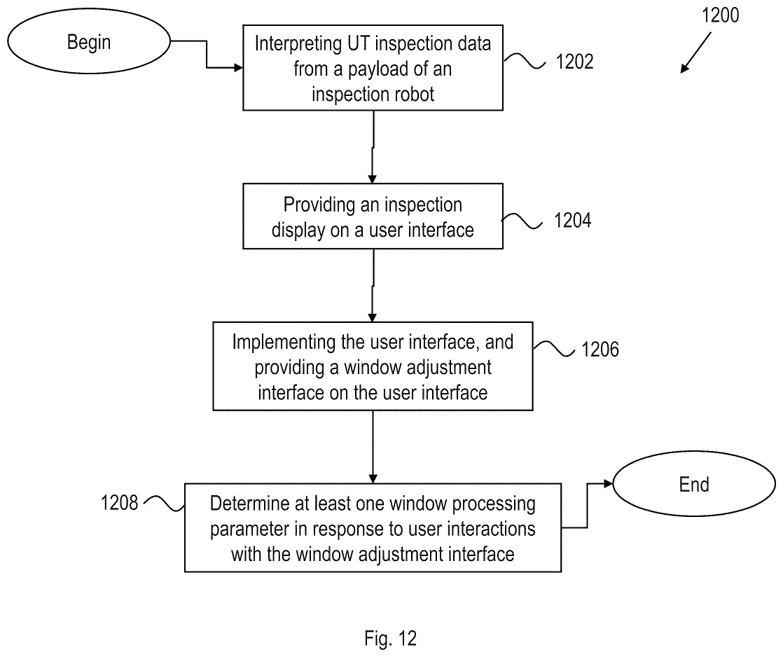

Referencing FIG. 12, an example procedure 1200 for determining window processing parameter(s) is schematically depicted. The example procedure 1200 includes an operation 1202 to interpret UT inspection data from a payload of an inspection robot, an operation 1204 to provide an inspection display on a user interface, an operation 1206 to implement the user interface, and to provide a window adjustment interface on the user interface, and an operation 1208 to determine at least one window processing parameter in response to user interactions with the window adjustment interface.

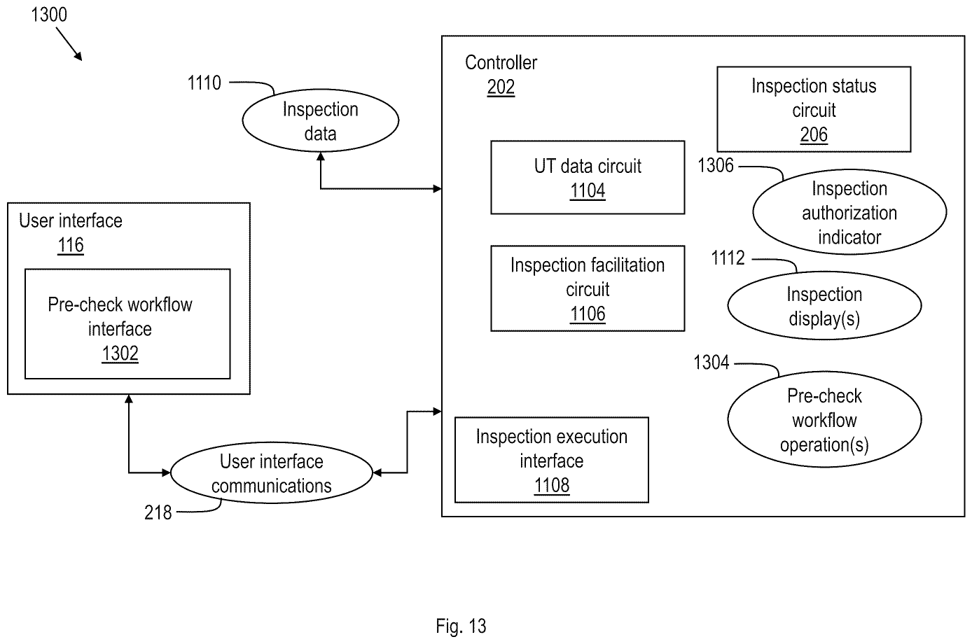

Referencing FIG. 13, an example apparatus 1300 includes a controller 202, that may be utilized with a system 100, and/or combined in whole or part: an inspection execution interface 1108 structured to implement a user interface 116 for an ultrasonic (UT) inspection by an inspection robot; an inspection facilitation circuit 1106 structured to provide an inspection display 1112 on the user interface 116, and to enforce a pre-check workflow operation 1304 in response to user interactions on the user interface 116 (e.g., implementing the pre-check workflow operation 1304 in a pre-check workflow interface 1302, for example provided on the user interface 116); and wherein the inspection facilitation circuit 1106 is further structured to provide an inspection authorization indicator 1306 in response to the pre-check workflow operation 1304. In certain embodiments, the inspection authorization indicator 1306 may be provided on a user interface, for example in a combined status indicator that blocks the inspection status indicator from being “green” or otherwise indicating that inspection operations can commence or continue until the pre-check workflow operations 1304 are complete. In certain embodiments, the inspection authorization indicator 1306 may be advisory - e.g., the operator can perform inspection operations with or without the indicator being set, for example when the apparatus 1300 operates to assist a cooperative operator in performing rapid, high quality, and complete inspection operations. In certain embodiments, the inspection authorization indicator 1306 may be enforced, for example where the controller 202 blocks or limits certain commands to the inspection robot 102 until the pre-check workflow operations 1304 are complete.

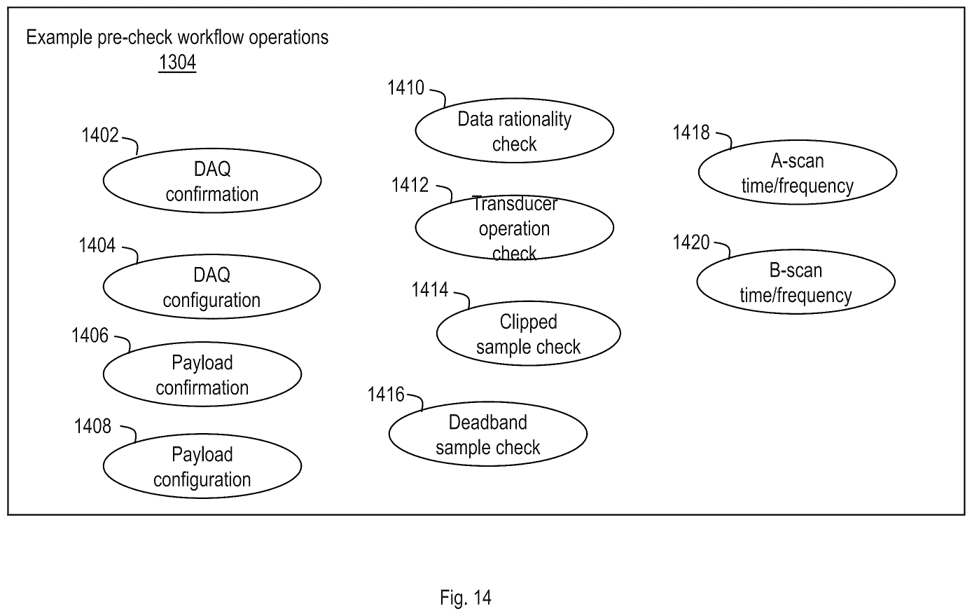

The example pre-check workflow operations 1304 can include any operations, best practices, actions required contractually and/or for regulatory purposes, or the like. In certain embodiments, pre-check workflow operations 1304 include operations to ensure that one or more of the DAQ, the payload, or the UT processing operations, are compatible with and properly configured for, the inspection operations. Referencing FIG. 14, example and non-limiting pre-check workflow operations 1304 include one or more of: a DAQ confirmation 1402 (e.g., ensuring the DAQ is appropriate for the payload, and configured to receive data properly); a DAQ configuration 1404 (e.g., providing an interface to configure the DAQ according to the payload and parameters of the inspection operation); a payload confirmation 1406 (e.g., ensuring that the payload, associated sensors, etc., are appropriate for the inspection operation, and calibrated for the parameters of the inspection operation); a payload configuration 1408 (e.g., displaying correct payload configuration and/or arrangement for the inspection operation, and/or providing an interface for the user to calibrate the payload, and ensure that the settings are correct for the inspection surface and inspection operations); a data rationality check 1410 (e.g., ensuring that impactors and/or transducers are responsive to commands and stimulus, and/or respond in the expected direction); a transducer operation check 1412 (e.g., ensuring that the transducers are receiving and have the appropriate settings, cutoff times, etc.); a clipped sample check 1414 (e.g., checking how many samples are clipped in the time domain); a deadband sample check 1416 (e.g., checking for operations outside of the deadband limit for the impactor and/or transducer); an A-scan check 1418 (e.g., a time domain and/or frequency domain check of the A-scan, which can illuminate certain data collection issues); and/or a B-scan check 1420 (e.g., a time domain and/or frequency domain check of the B-scan, with a number of stacked A-scan, which can illuminate certain data collection issues).

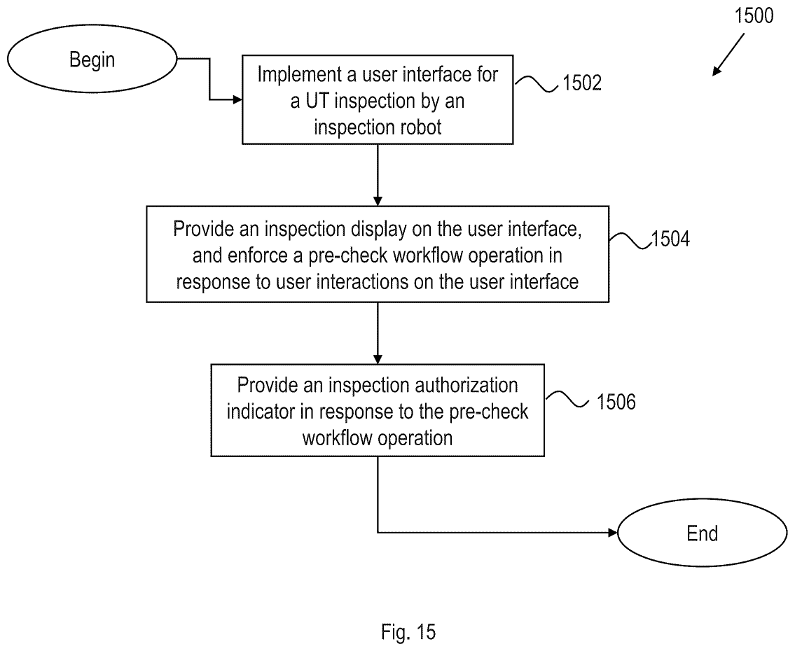

Referencing FIG. 15, an example procedure 1500 for determining an inspection authorization indicator is schematically depicted. The example procedure 1500 includes an operation 1502 to implement a user interface for a UT inspection by an inspection robot, an operation 1504 to provide an inspection display on the user interface, and to enforce a pre-check workflow operation in response to user interactions on the user interface, and an operation 1506 to provide an inspection authorization indicator in response to the pre-check workflow operation.

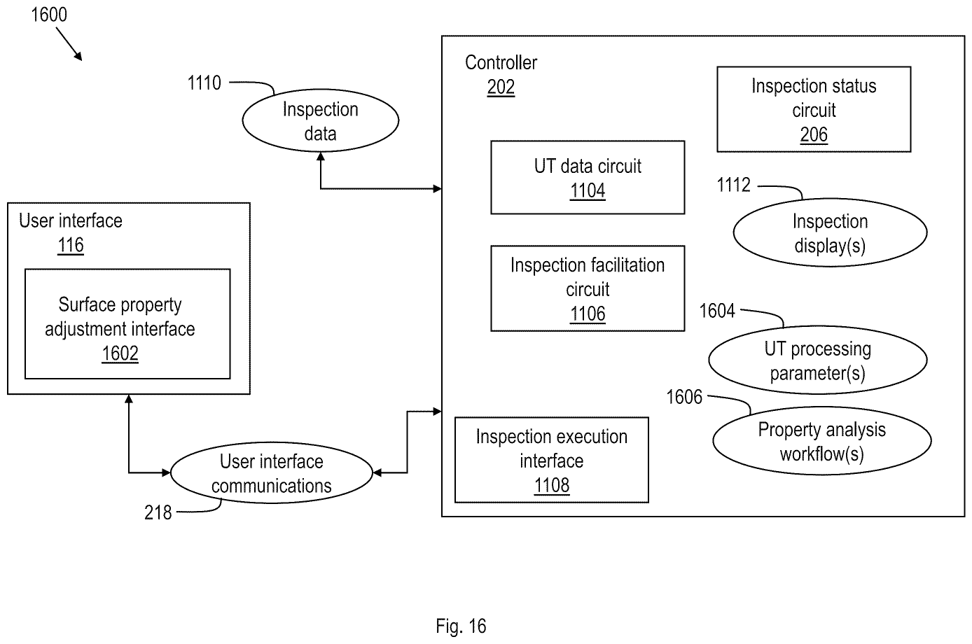

Referencing FIG. 16, an example apparatus 1600 includes a controller 202, that may be utilized with a system 100, and/or combined in whole or part: an ultrasonic (UT) data circuit 1104 structured to interpret UT inspection data 1110 from a payload of an inspection robot, the payload including an ultrasonic (UT) sensor; an inspection facilitation circuit 1106 structured to provide an inspection display 1112 on a user interface 116; an inspection execution interface 1108 structured to implement the user interface 116, to provide a surface property adjustment interface 1602 on the user interface 116, and to determine at least one UT processing parameter 1604 in response to user interactions with the surface property adjustment interface 1602. Example and non-limiting UT processing parameters include one or more of: a surface velocity parameter, a concrete velocity parameter, and/or a P-wave velocity parameter. An example inspection facilitation circuit 1106 enforces a property analysis workflow 1606 in response to user interactions 116 on the user interface, for example to ensure that the operator provides and considers the appropriate settings for the UT processing parameters before commencing or continuing an inspection operation. In certain embodiments, the property analysis workflow 1606 may be advisory, or may be compulsory. In certain embodiments, the property analysis workflow 1606 may be performed before the inspection operations, during inspection operations (e.g., between runs), and/or after inspection operations (e.g., during post-processing operations, and/or preparing an inspection report). An example property analysis workflow 1606 determines a median velocity value from a filtered inspection data set, resulting in a representative velocity value for the substrate that is likely to provide high quality inspection data for the inspection surface.

An example property analysis workflow 1606 includes one or more operations such as: processing some UT inspection data from the surface (e.g., sampling data to be utilized to confirm and/or adjust the UT processing parameters); adjusting projection modes of the impactors to enhance visual clarity; selecting the highest mass impactor to perform testing operations; verify the nominal thickness of the asset (e.g., utilizing a characteristic thickness from the frequency information); filtering the sampled data to exclude outliers (e.g., greater than +/−1″ from the nominal thickness); accessing a correlation plot to analyze relationships between points); adjusting a metric filter slider (e.g., percentage of good/bad points for display, according to experience on the asset and/or guidelines, for example 60%); identifying correlated areas (e.g., finding areas of interest, areas expected to have the same acoustic environment, etc.); review the concrete velocity plot (e.g., select a concrete velocity plot, or CPP, for further analysis); document a median velocity value within the selected region of the CPP plot (e.g., this value can be used for this region, and/or for correlated regions); and/or update the configuration (e.g., modify the parameter using the interface, for example setting the concrete velocity or P-wave velocity).

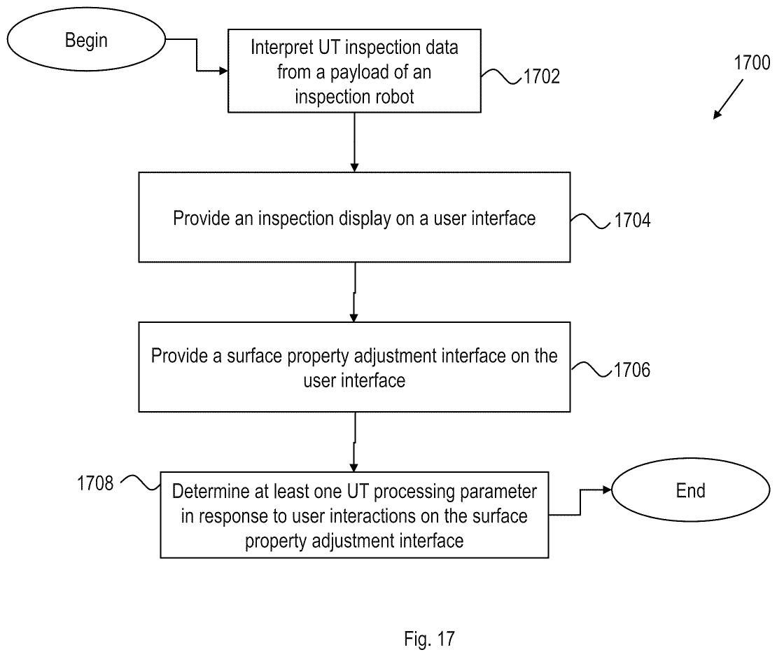

Referencing FIG. 17, an example procedure 1700 includes an operation 1702 to interpret UT inspection data from a payload of an inspection robot, an operation 1704 to provide an inspection display on a user interface, an operation 1706 to provide a surface property adjustment interface on the user interface, and an operation 1708 to determine at least one UT processing parameter in response to user interactions on the surface property adjustment interface.

Referencing FIG. 16, an example apparatus 1600 includes a controller 202, that may be utilized with a system 100, and/or combined in whole or part: an ultrasonic (UT) data circuit 1104 structured to interpret UT inspection data 1110 from a payload of an inspection robot, the payload including an ultrasonic (UT) sensor; an inspection facilitation circuit 11-6 structured to provide an inspection display 1112 on a data analysis parameter interface 1802; an inspection execution interface 1108 structured to implement the data analysis parameter interface 1802, and to determine at least one selected data value 1804, and at least one UT processing parameter 1604, in response to user interactions with the data analysis parameter interface 1802. An example inspection execution interface 1108 allows the user to select a point or a range of points, and to display the at least one UT processing parameter associated therewith. For example, the user may select a range of points graphically (e.g., bounding a region of interest, selecting a feature of interest, selecting a pre-identified region, etc.), analytically (e.g., searching and/or filtering for data points of interest, for example based on the data, job time, asset location, etc.), through a menu and/or from a shared view by another user, or the like. An example inspection execution interface allows the user to adjust the at least one UT processing parameter, where the inspection facilitation circuit 1106 updates the inspection display in response to the adjusted at least one UT processing parameter (e.g., displaying inspection results, inspection quality values, etc., based on processing the inspection data according to the adjusted UT processing parameter(s)). In certain embodiments, the adjusted UT processing parameter(s) lead to a difference in identified features (e.g., physical structures, changes in some aspect of the surface, areas near damage, areas of importance and/or high wear regions, etc.), where the inspection facilitation circuit 1106 updates the inspection display by providing an updated feature map for the inspection surface. An example inspection facilitation circuit 1106 updates the inspection display with an updated inspection data quality value. Example and non-limiting UT process parameters include one or more of: an initiating time value, a time delay value, a characteristic thickness value, a sound velocity value, a clipping limit value, or a deadband limit value.

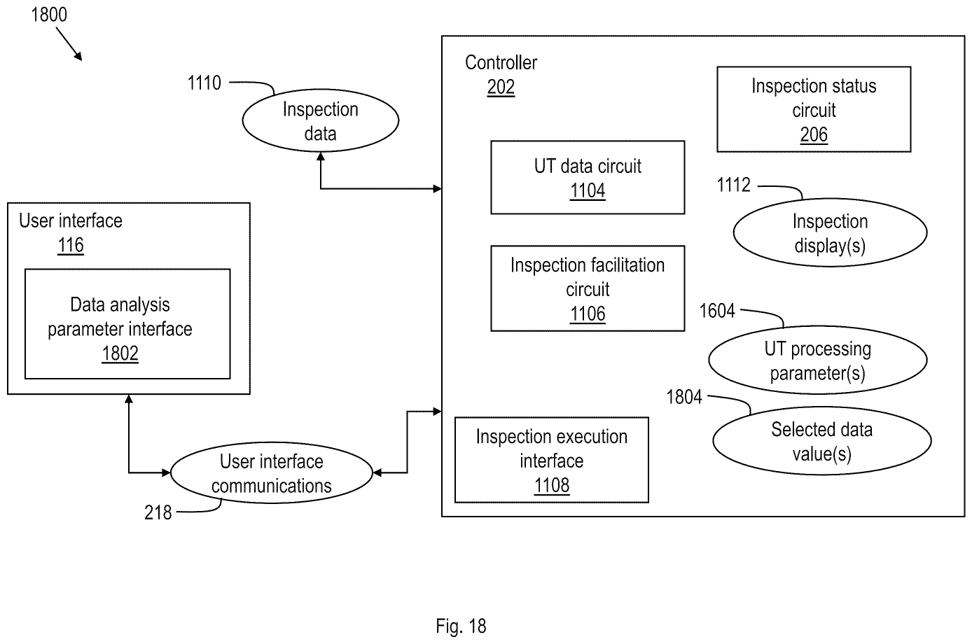

An example inspection facilitation circuit 1106 updates the inspection display by providing a comparison display on the data analysis parameter interface in response to the at least one UT processing parameter. An example inspection facilitation circuit 1106 updates the inspection display by applying the adjusted UT processing parameter(s) to a selected region of the inspection surface. An example inspection facilitation circuit 1106 updates the inspection display by applying the adjusted UT processing parameter(s) to a selected range of inspection data values. The example apparatus 1800 of FIG. 18 allows a user to inspect the UT analysis for any data values, regions, and/or areas of interest, to see the effects of various processing scenarios, and to quickly apply any adjustments to the processing operations after previewing the effects of adjustments.

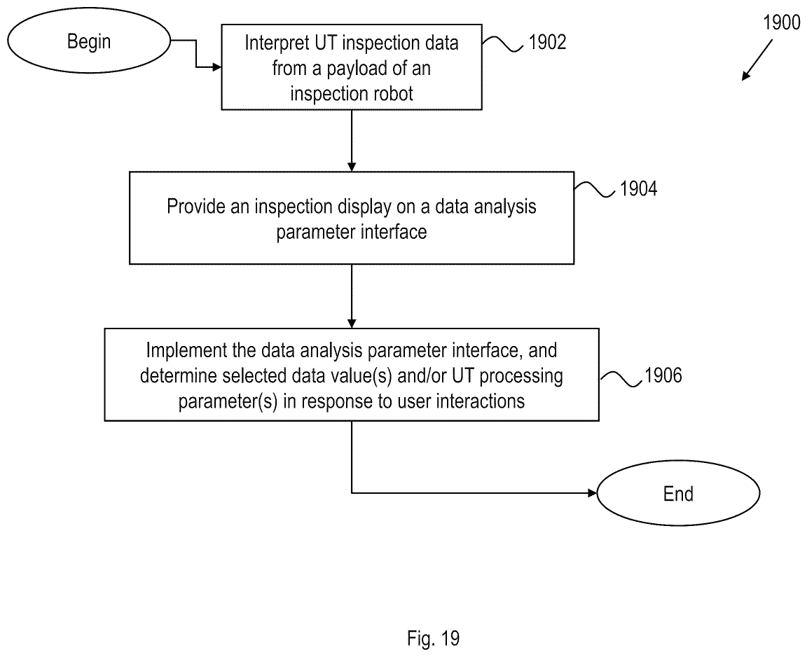

Referencing FIG. 19, an example procedure 1900 for implementing a data analysis parameter interface is schematically depicted. The example procedure 1900 includes an operation 1902 to interpret UT inspection data from a payload of an inspection robot, an operation 1904 to provide an inspection display on a data analysis parameter interface, and an operation 1906 to implement the data analysis parameter interface, and determine selected data value(s) and/or UT processing parameters in response to user interactions.

In some aspects, the techniques described herein relate to a method, including: interpreting UT inspection data from a payload of an inspection robot, the payload including an ultrasonic (UT) sensor; providing an inspection display on a data analysis parameter interface; implementing the data analysis parameter interface, and determining at least one selected data value and at least one UT processing parameter, in response to user interactions with the data analysis parameter interface.

Referencing FIG. 20, an example phase plot 2002 and dispersion plot 2004 are depicted as illustrative data. The example phase plot 2002 may be generated from a cross-spectral density function on a digital signal plot (e.g., FIG. 6) to generate a phase plot (e.g., FIG. 20), which can then be masked (e.g., masking portions 2006, 2010 are not utilized for the dispersion plot 2004), wherein the primary phase progressions in the phase plot (e.g., well behaved phase progression, and complete through the available range of phase). The dispersion plot 2004 indicates the slope of the active phase plot graph (e.g., the portion 2008 that is not masked 2006, 2010) by depth, which can be utilized to display the sound velocity by depth, and which is useful in identifying cracks, voids, honeycombs, etc., especially in combination with other inspection data (e.g., bulk depth). In certain embodiments, the shape or progression of the dispersion plot 2004, and/or discontinuities therein, can be utilized to provide data verification, determine quality values, identify features of interest, and/or identify the type or severity of anomalies or detected inspection features.

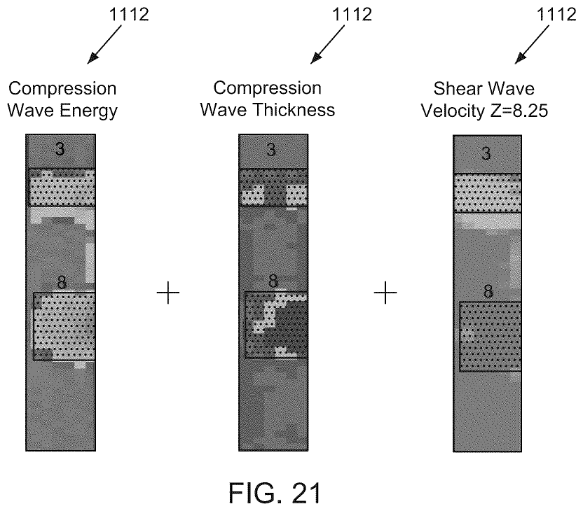

Referencing FIG. 21, example and non-limiting inspection displays 1112 are depicted, for example showing the operator the status of the inspection operations, high level results, identified features, and/or data verification issues that may be present. The example of FIG. 21 includes a compression wave energy plot (e.g., signal energy returned to the transducer, for example determined by integrating a compression wave amplitude), a compression wave thickness plot (e.g., a characteristic thickness determined by the compression wave primary frequency), and a shear wave velocity (e.g., which should match estimated properties where the inspection surface is nominal and undamaged).

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems herein. The terms computer, computing device, processor, circuit, and/or server, (“computing device”) as utilized herein, should be understood broadly.

An example computing device includes a computer of any type, capable to access instructions stored in communication thereto such as upon a non-transient computer readable medium, whereupon the computer performs operations of the computing device upon executing the instructions. In certain embodiments, such instructions themselves comprise a computing device. Additionally or alternatively, a computing device may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware and/or computing devices include, without limitation, a general-purpose computer, a server, an embedded computer, a mobile device, a virtual machine, and/or an emulated computing device. A computing device may be a distributed resource included as an aspect of several devices, included as an interoperable set of resources to perform described functions of the computing device, such that the distributed resources function together to perform the operations of the computing device. In certain embodiments, each computing device may be on separate hardware, and/or one or more hardware devices may include aspects of more than one computing device, for example as separately executable instructions stored on the device, and/or as logically partitioned aspects of a set of executable instructions, with some aspects comprising a part of one of a first computing device, and some aspects comprising a part of another of the computing devices.

A computing device may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer-to-peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information (“receiving data”). Operations to receive data include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first receiving operation may be performed, and when communications are restored an updated receiving operation may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g., where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The methods and/or processes described above, and steps thereof, may be realized in hardware, program code, instructions, and/or programs or any combination of hardware and methods, program code, instructions, and/or programs suitable for a particular application. The hardware may include a dedicated computing device or specific computing device, a particular aspect or component of a specific computing device, and/or an arrangement of hardware components and/or logical circuits to perform one or more of the operations of a method and/or system. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above, and combinations thereof, may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer readable instructions described above. All such permutations and combinations are intended to fall within the scope of the present disclosure.

While the disclosure has been disclosed in connection with certain embodiments shown and described in detail, various modifications and improvements thereon will become readily apparent to those skilled in the art. Accordingly, the present disclosure is not to be limited by the specific examples described and depicted, but is to be understood in the broadest sense allowable by law.

1. A system, comprising:
an inspection robot comprising a payload configured to interrogate an inspection surface in response to the inspection robot moving on the inspection surface, the payload comprising an ultrasonic (UT) sensor;
a data verification controller, comprising:
a verification data circuit structured to interpret verification data from the payload;
an inspection status circuit structured to determine an inspection data quality value in response to the verification data; and
a data verification circuit structured to perform a data verification operation in response to the inspection data quality value.

2. The system of claim 1, wherein the inspection status circuit is further structured to determine the inspection data quality value by determining a bonding value.

3. The system of claim 1, wherein the inspection status circuit is further structured to determine the inspection data quality value by determining a noise amplitude value.

4. The system of claim 1, wherein the inspection status circuit is further structured to determine the inspection data quality value by determining a P-wave integral value.

5. The system of claim 1, wherein the inspection status circuit is further structured to determine the inspection data quality value in response to a correlation map.

6. The system of claim 1, wherein the inspection status circuit is further structured to determine the inspection data quality value in response to a characteristic thickness value.

7. The system of claim 1, wherein the inspection status circuit is further structured to determine the inspection data quality value in response to a spectral analysis of the verification data.

8. The system of claim 7, wherein the spectral analysis further comprises a dispersion plot.

9.-26. (canceled)

27. A method, comprising:
interrogating an inspection surface with an ultrasonic (UT) sensor;
interpreting verification data from the interrogating;
determining an inspection data quality value in response to the verification data; and
performing a data verification operation in response to the inspection data quality value.

28. The method of claim 27, wherein determining the inspection data quality value comprises determining a bonding value.

29. The method of claim 27, wherein determining the inspection data quality value comprises determining a noise amplitude value.

30. The method of claim 27, wherein determining the inspection data quality value comprises determining a signal to noise ratio value.

31. The method of claim 27, wherein determining the inspection data quality value comprises determining a P-wave integral value.

32. The method of claim 27, further comprising determining the inspection data quality value in response to a correlation map.

33. The method of claim 32, further comprising determining the correlation map in response to a differential gradient of the verification data.

34. The method of claim 32, further comprising determining the inspection data quality value in response to a characteristic thickness value.

35. The method of claim 27, further comprising determining the inspection data quality value in response to a spectral analysis of the verification data.

36. The method of claim 35, further comprising performing the spectral analysis by determining a dispersion plot.

37. The method of claim 27, wherein performing the data verification operation comprises labeling a range of data values with an inspection data value.

38. The method of claim 27, wherein performing the data verification operation comprises providing an inspection status indicator on a user interface.

39.-100. (canceled)
